(19) United States
(12) Patent Application Publication
(10) Pub. No.: US 2021/0117867 A1
(43) Pub. Date: Apr. 22, 2021

(54) METHOD AND APPARATUS FOR SUBTYPING SUBJECTS BASED ON PHENOTYPIC INFORMATION

(71) Applicant: Oxford University Innovation Limited, Oxford (GB)

(72) Inventors: Andrew David CLIFTON, Oxford (Oxfordshire) (GB); Nazli FARAJIDAVAR, Oxford (Oxfordshire) (GB); Tingting ZHU, Oxford (Oxfordshire) (GB); Xiaorong DING, Oxford (Oxfordshire) (GB); Peter WATKINSON, Oxford (Oxfordshire) (GB)

(21) Appl. No.: 17/051,795

(22) PCT Filed: Mar. 12, 2019
(86) PCT No.: PCT/GB2019/050682

§ 371 Date: Oct. 30, 2020

(30) Foreign Application Priority Data
May 3, 2018 (GB) 1807308.0

(51) Int. Cl. G06N 20/10; G16B 40/30; G16B 40/20; G16H 50/20; G16H 50/70

(52) U.S. Cl. CPC G06N 20/10; G16B 40/30; G16H 50/70; G16H 50/20; G16B 40/20

(57) ABSTRACT

Methods and apparatus for subtyping subjects based on phenotypic information are disclosed. In one arrangement, a data receiving unit receives a subject data unit for each of a plurality of subjects. Each subject data unit represents a plurality of different phenotypic information items about the subject. A data processing unit uses a deep learning algorithm to derive a lower dimensional representation of each subject data unit and a clustering algorithm to detect clusters of the resulting lower dimensional representations. The deep learning algorithm and clustering algorithm are implemented by a single mathematical model in which the derivation of the lower dimensional representations and the detection of the clusters are performed jointly.

Embodiments of the disclosure relate to subtyping subjects according to phenotypic information, particularly in the case where the phenotypic information is multidimensional.

It is desirable to classify subjects into phenotypic groups to improve treatment and/or risk management. Detecting phenotypic subgroups of patients suffering from complex diseases such as Parkinson's disease (PD) and Chronic Obstructive pulmonary disease (COPD), for example, can allow stratified risk assessment. Furthermore, it can provide support for early detection of deteriorating patients, determination of individualized and customized treatment, and prevention strategies for different phenotypic groups, which ultimately results in enhanced treatment outcome. There would also be significant value for understanding patient phenotypes for improving treatments, conducting clinical trials, etc.

It is an object of the invention to provide improved methods and apparatus for identifying phenotypic groups of subjects.

According to an aspect of the invention, there is provided a computer-implemented method of subtyping subjects based on phenotypic information, comprising: receiving a subject data unit for each of a plurality of subjects, each subject data unit representing a plurality of different phenotypic information items about the subject of the subject data unit; using a deep learning algorithm to derive a lower dimensional representation of each subject data unit and a clustering algorithm to detect clusters of the resulting lower dimensional representations, each cluster representing a subtype of subjects that are phenotypically related to each other, wherein: the deep learning algorithm and clustering algorithm are implemented by a single mathematical model in which the derivation of the lower dimensional representations and the detection of the clusters are performed jointly.

Thus, a method is provided in which a deep learning algorithm and clustering algorithm are implemented in a joint framework. This allows the process of determining representations of high-dimensional features in the input data (the subject data units) to inform the clustering process and vice versa, which the inventors have found significantly improves performance relative to alternative approaches in which clustering is performed without dimension reduction or where dimension reduction and clustering are performed completely separately. The improved performance allows subjects to be clustered into groups more meaningfully and efficiently, thereby enabling management of subjects (e.g. risk management, treatment plan selection, etc.) to be performed more reliably and/or more efficiently.

In an embodiment, the joint performance of the derivation of the lower dimensional representations and the detection of the clusters comprises optimizing a unified loss function having a term corresponding to the derivation of the lower dimensional representations and a term corresponding to the detection of the clusters, optionally with a regularization term. The inventors have found that performing the joint optimization based on a unified loss function can be implemented particularly efficiently.

According to an alternative aspect, there is provided an apparatus an apparatus for subtyping subjects based on phenotypic information, comprising: a data receiving unit configured to receive a subject data unit for each of a plurality of subjects, each subject data unit representing a plurality of different phenotypic information items about the subject of the subject data unit; and a data processing unit configured to: use a deep learning algorithm to derive a lower dimensional representation of each subject data unit and a clustering algorithm to detect clusters of the resulting lower dimensional representations, each cluster representing a subtype of subjects that are phenotypically related to each other, wherein: the deep learning algorithm and clustering algorithm are implemented by a single mathematical model in which the derivation of the lower dimensional representations and the detection of the clusters are performed jointly.

Embodiments of the invention will now be described, by way of example only, with reference to the accompanying drawings in which corresponding reference symbols indicate corresponding parts, and in which:

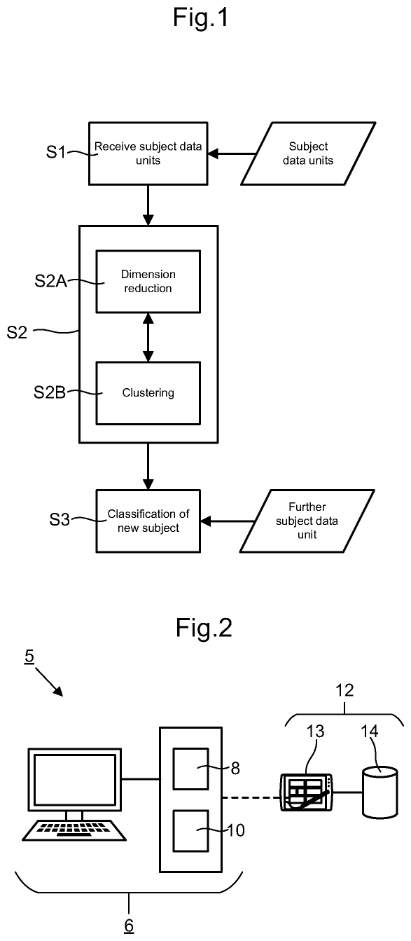

FIG. 1 is a flowchart depicting a method of subtyping subjects based on phenotypic information according to an embodiment;

FIG. 2 schematically depicts an apparatus for implementing methods of the type depicted in FIG. 1;

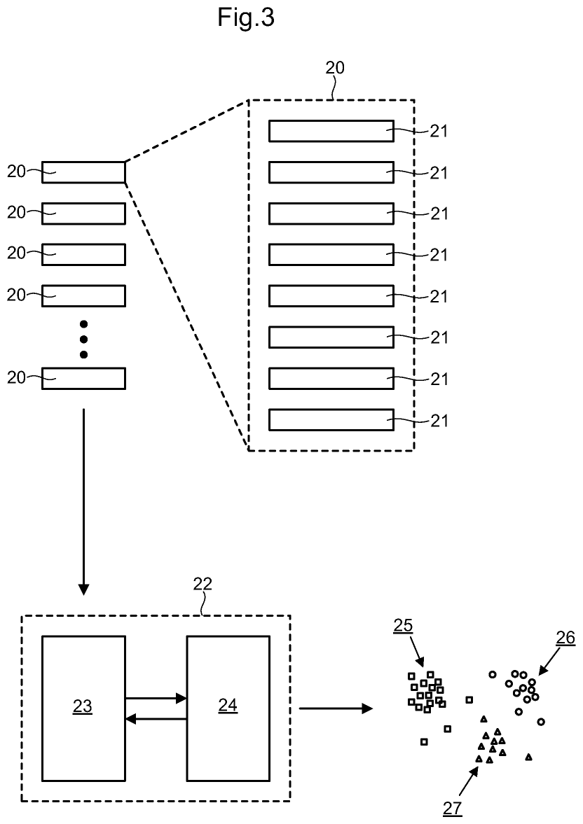

FIG. 3 schematically depicts elements of the method of FIG. 1;

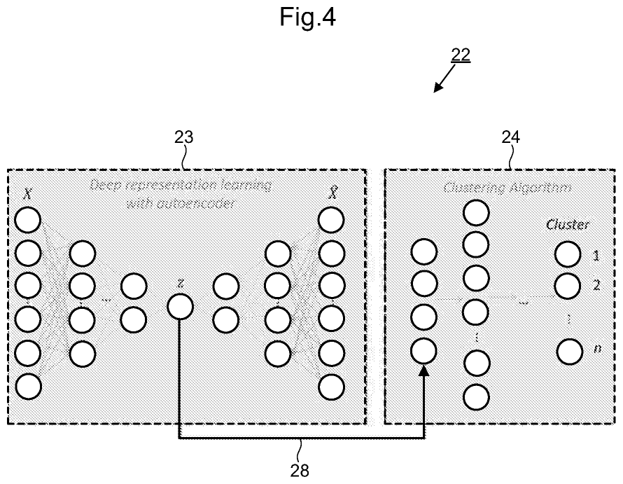

FIG. 4 schematically depicts example configurations for a single mathematical model comprising a deep learning algorithm and a clustering algorithm;

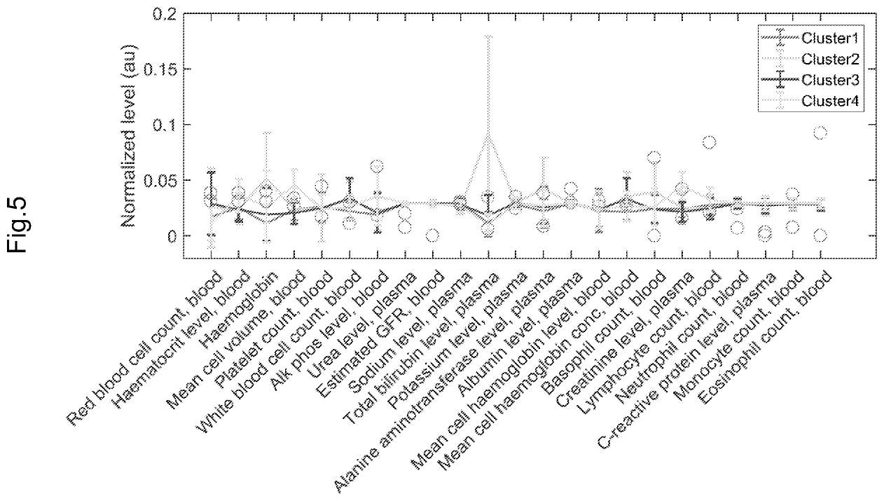

FIG. 5 depicts a normalized level of 23 blood test variables for different clusters of subject data units, in which error bars represent mean and standard deviation of the normalized level, and circles represent normal high and low range of each variable; and

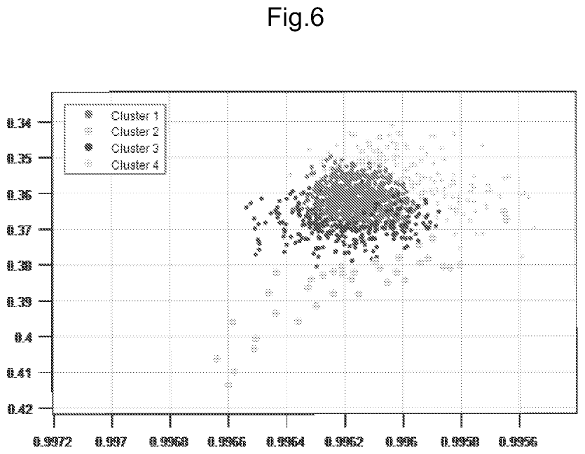

FIG. 6 depicts a 2D representation of the 23D normalized blood test variables clustered using the method of FIG. 1.

Methods of the present disclosure are computer-implemented. Each step of the disclosed methods may therefore be performed by a computer. The computer may comprise various combinations of computer hardware, including for example CPUs, RAM, SSDs, motherboards, network connections, firmware, software, and/or other elements known in the art that allow the computer hardware to perform the required computing operations. The required computing operations may be defined by one or more computer programs. The one or more computer programs may be provided in the form of media, optionally non-transitory media, storing computer readable instructions. When the computer readable instructions are read by the computer, the computer performs the required method steps. The computer may consist of a self-contained unit, such as a general-purpose desktop computer, laptop, tablet, mobile telephone, smart device (e.g. smart TV), etc. Alternatively, the computer may consist of a distributed computing system having plural different computers connected to each other via a network such as the internet or an intranet.

As explained in the introductory part of the description, it is desirable to subtype (which may also be referred to as group, cluster or classify) subjects into phenotypic subtypes (groups, clusters or classes) to improve treatment and/or risk management. The following detailed description provides example approaches for achieving this in an efficient way. The methods disclosed can be provided as part of a pipeline involving data curation and pre-processing (cleaning, imputation, and feature selection), as well as the clustering methods described specifically below with reference to the figures. The clustering methods disclosed can be used to allow accurate identification of phenotypic subtypes in patient cohorts for complex disease, which can be used for example to stratify patients with complex diseases into subtypes with differing disease progression and risk of disease complications. The sub-stratification of the diseases makes it possible to more efficiently screen risk factors (genetic or/and environmental) and/or tailor and target early treatment to patients, thereby enabling a route towards precision medicine and associated improvements in healthcare delivery and patient outcomes.

FIG. 1 schematically depicts a framework in flowchart form for a method of subtyping subjects (e.g. human or animal subjects) based on phenotypic information. The method may be performed by an apparatus 5 as depicted in FIG. 2. FIG. 3 provides a visualisation of aspects of the method. The terms “human or animal subject” or “subject” may be used interchangeably with the term “patient” in the following description.

In an embodiment, the method comprises a step S1 of receiving a subject data unit 20 for each of a plurality of subjects. Thus, a set comprising a plurality of subject data units 20 is received, as depicted schematically in the top left of FIG. 3. Each subject data unit 20 represents a plurality of different phenotypic information items 21 (e.g. measurement data values) about the subject of the subject data unit 20. Each of the phenotypic information items 21 represents a dimension of the subject data unit 20. Thus, if 33 different items are present, the subject data unit has 33 dimensions. The plural phenotypic information items 21 of one of the subject data units 20 is depicted schematically in FIG. 3. In an embodiment, the phenotypic information items comprise one or more of the following: blood markers, genetic data, clinical data, medical imaging data (including neuroimaging data), demographic data, age, gender, comorbidity, disease development information, medication information, drug response/reaction information, blood test information. In other embodiments, other phenotypic information items may be provided. Some or all of the phenotypic information items may be provided by an Electronic Health Record (EHR). In an embodiment, as described below with reference to FIG. 5, the phenotypic information items comprise one or more (or all) of the following laboratory tests: red blood cell count in blood, haematocrit level in blood, haemoglobin level in blood, mean cell volume in blood, platelet count in blood, white blood cell count in blood, Alk phos level in blood, urea level in plasma, estimated GFR in blood, sodium level in plasma, total bilirubin level in plasma, potassium level in plasma, alanine aminotransferase level in plasma, albumin level in plasma, mean cell haemoglobin level in blood, mean cell haemoglobin concentration in blood, basophil count in blood, creatinine level in plasma, lymphocyte count in blood, neutrophil count in blood, c-reactive protein level in plasma, monocyte count in blood, eosinophil count in blood. More generally, the phenotypic information items may relate to any observable (measurable) characteristic of the subject.

In step S2A, a deep learning algorithm 23 is used to derive a lower dimensional representation of each subject data unit 20 (i.e. having lower dimensions than the original subject data unit 20). In step S2B, a clustering algorithm 24 is used to detect clusters 25-27 (see FIG. 3) of the resulting lower dimensional representations. Each cluster 25-27 represents a subtype (group, cluster or class) of subjects that are phenotypically related to each other. The deep learning algorithm 23 and clustering algorithm 24 are implemented by a single mathematical model 22 in which the derivation of the lower dimensional representations and the detection of the clusters are performed (optimized) jointly. Steps S2A and S2B thus form a single combined dimension reducing and clustering step S2.

Exemplary configurations for the single mathematical model 22 are now described in further detail with reference to FIG. 4.

In an embodiment, the mathematical model 22 is configured so that the clustering algorithm 24 provides supervisory signals to the deep learning algorithm 23. In a particular example described below, the deep learning algorithm 23 is an autoencoder (AE) deep representation learning algorithm and the clustering algorithm 24 is an unsupervised Gaussian Mixture Model (GMM) clustering model.

FIG. 4 depicts an illustrative structure of an AE based deep representation learning algorithm 23 on the left. In this algorithm 23, the original high-dimensional data X (representing the input subject data units 20) is transformed into a lower-dimensional representation Z. To obtain the most powerful representation of X, a deep neural network (NN) with m layers may be provided, with nₘ nodes per layer. Taking AE as an example, the deep learning algorithm 23 may comprise an encoder and a decoder, wherein the encoder works to extract a code of the input, while the decoder produces the output using the code. The goal is to get an output identical with the input, such that the latent feature Z can best preserve the key information of the input X.

To achieve the above goal, the NN may be trained with a loss function L_{d}(X, \hat{X}):

$${L}_{d} ( X , \hat{X} ) = \frac{1}{N} {\sum }_{i = 1}^{N} L ( {x}_{i} , {\hat{x}}_{ι}^{′} )$$

where L(xᵢ, \hat{x}ₗ′) is the loss function that characterizes the reconstruction error caused by the deep AE in the compression network. The loss function may comprise the root mean square error or another error metric. It is desirable to achieve the lowest reconstruction error possible to ensure the low-dimensional representation contains as much of the information present in the high-dimensional data as possible.

After the dimensional reduction by the deep learning algorithm 23, the latent feature Z is fed (arrow 28) to the clustering algorithm 24. In an embodiment, clustering algorithm 24 is parametric model-based (e.g. GMM) or nonparametric (such as hierarchical clustering). GMM is used as an exemplary clustering algorithm 24 for the following description. It is understood that the GMM could be replaced by a different clustering algorithm 24.

In the GMM setting, we assume the investigated heterogeneous sample Z has finite mixture of multivariate normal densities:

$$g ( Z ; θ ) = {\sum }_{k = 1}^{K} {π}_{k} Ø ( Z ; {θ}_{k} ) where \text{:}$$

$$Ø ( Z ; {θ}_{k} ) = \frac{1}{2 {π}^{p / 2} {\vert {\sum }_{k} \vert}^{1 / 2}} {exp}^{- ( \frac{1}{2} {( Z - {μ}_{k} )}^{T} {Σ}_{k}^{- 1} ( Z - {μ}_{k} ) )}$$

is the multivariate Gaussian density with θₖ=(μₖ,Σₖ), K the number of the clustering components, and πₖ the proportions of the k^{th} component, μₖ, Σₖ are the mean and covariance of data belonging to the k^{th} components.

To learn the parameters, i.e. θₖ, Σₖ, a well-established algorithm—Expectation-Maximization Algorithm (EM algorithm) can be applied to update the parameters. As the name indicates, there are two steps in this algorithm: the expectation step and the maximization step. In the expectation step, the probability \hat{γ}=softmax(p), i.e. the cluster membership matrix which assigns the portion of data to be part of the k^{th} cluster, can be computed. In the maximization step, the parameters πₖ, μₖ, Σₖ are updated as:

$${\hat{φ}}_{k} = {\sum }_{i = 1}^{N} \frac{{\hat{γ}}_{ik}}{N} , {\hat{μ}}_{k} = \frac{{\sum }_{i = 1}^{N} {\hat{γ}}_{ik} {z}_{i}}{{\sum }_{i = 1}^{N} {\hat{γ}}_{ik}} , {\hat{\sum }}_{k} = \frac{{\sum }_{i = 1}^{N} {\hat{γ}}_{ik} ( {z}_{i} - {\hat{μ}}_{k} ) {( {z}_{i} - {\hat{μ}}_{k} )}^{T}}{{\sum }_{i = 1}^{N} {\hat{γ}}_{ik}}$$

The optimal parameters can then be obtained through the minimization of the negative likelihood of the model:

L_{c}(Z,θ_{c})=Σᵢ₌₁^{N} log(Σᵢ₌₁^{K}φₖØ(Z;θₖ))

The proposed joint framework combines the abovementioned deep representation learning and the clustering into a single model with a unified loss function:

U(θ_{d},θ_{c})=λ_{d}L_{d}(X,\hat{X})+λ_{c}L_{c}(Z,θ_{c})+λᵣLᵣ

where the L_{d}(x, \hat{X}) is the loss function of the dimensionality reduction, L_{c}(Z,θ_{c}) the loss function for the clustering, Lᵣ the regulation item, and the λ_{d},λ_{c},λᵣ are the hyperparameters that can make the unified loss function work best. Thus, the joint performance of the derivation of the lower dimensional representations and the detection of the clusters may comprise optimizing a unified loss function having a term corresponding to the derivation of the lower dimensional representations (L_{d}) and a term corresponding to the detection of the clusters (L_{c}), optionally with a regularization term (Lᵣ).

By optimizing the unified loss function with a number of iterations of training of the deep learning algorithm 23 as well as the clustering algorithm 24, it is possible to obtain not only more powerful feature representations, but also precise assignment of data into corresponding clusters.

In step S3, a further subject data unit is obtained. The further subject data unit comprises a plurality of different phenotypic information items about a subject to be assessed. The further subject data unit may take any of the forms described above for the other subject data units. The single mathematical model 22 is used to derive a lower dimensional representation of the subject data unit and assign the lower dimensional representation of the subject data unit to one of the detected clusters 25-27, thereby identifying to which of the clusters the subject to be assessed belongs. Thus, steps S1-S2 effectively train the method by generating clusters of subject data units from reference subjects. A subject data unit from a new subject can then be processed to determine which of the clusters the new subject belongs to, thereby subtyping the new subject.

Aspects of the above-described methods may be implemented by an apparatus 5 such as that depicted in FIG. 2. In this particular example, the apparatus 5 can perform measurements using a sensor system 12. The sensor system 12 may comprise a local electronic unit 13 (e.g. a tablet computer, smart phone, smart watch, etc.) and a sensor unit 14 (e.g. a blood pressure monitor, heart rate monitor, etc.). The measurements may comprise one or more vital signs measurements, including one or more of the following: blood pressure measurements (e.g. systolic blood pressure, SBP), heart rate measurements, breathing rate measurements, temperature measurements, oxygen saturation measurements. Alternatively, the measurement may comprise analysis of samples taken from subjects (e.g. measurements of blood samples, medical images, etc.). The measurements performed by the sensor system 12 may provide one or more of the phenotypic information items 21 of one or more of the subject data units 20. A data receiving unit 8 is provided that receives the subject data units 20 (either from the sensor system 12 or from another source, such as a storage means or data connection to an intranet or internet). In an embodiment, the data receiving unit 8 receives data from an Electronic Health Record (EHR). The data receiving unit 8 may form part of a computing system 6 (e.g. laptop computer, desktop computer, etc.). The computing system 6 may further comprise a data processing unit 10 configured to carry out steps of the method.

An exemplary application of a method of an embodiment to identify subtypes of Parkinson's Disease (PD) is now described. PD is a typical complex and heterogeneous disease. In this example, the deep learning algorithm 23 is an autoencoder (AE) and the clustering algorithm 24 is an unsupervised Gaussian Mixture Model (GMM) clustering model. The phenotypic information items 21 comprise 23 laboratory test items in this example (mainly blood biomarkers, but other information such as neuroimaging, genetic, clinical, medical imaging, demographic, and so on could be used in extensions of the example), such that each subject data unit 20 has 23 dimensions. The laboratory test items correspond to the first laboratory assessment of the patient and are commonly prescribed as an initial health assessment indicator in this area. The AE deep learning algorithm 23 was used to extract the abstract representations of the 23-dimensional variables by transforming the 23D variables into 3D, which is then feed to the GMM clustering algorithm 24 to update the clusters.

FIG. 5 outlines the mean and standard deviations of the normalization level of 23 blood test items. The original blood test items have various units, and they have been normalized for better processing and visualization. We might observe that cluster 2 has significant higher mean level and variance than other clusters in terms of haemoglobin and total bilirubin level of plasma, suggesting the different disease manifestations compared with other clusters. It can be readily seen from FIG. 5 that it is difficult to discriminate the four clusters with all 23D phenotypic (blood test) information items as the mean and the standard deviation of the clusters are highly overlapping. Application of the algorithms 23 and 24 of the present method, however, allow the clusters to be clearly separated and observed from each other by effectively projecting the 23D data into a 3D space. FIG. 6 depicts a 2D projection of the 3D space to allow visualisation of the clustering. It can be seen from FIG. 6 that the four clusters are distinct and separable from each other.

With further analysis of the clusters identified by the method (representing subtypes of the complex disease in this example), the inventors found that each subtype represents a different stage of the disease progression, and the subpopulation of each subtype features similar clinical manifestations. All those findings could provide guidance for treatment decisions of a given individual. If the subtype is found to have causal and clinically justified association with underlying mechanism, it can serve as an automated mechanism for understanding the aetiology of the disease.

1. A computer-implemented method of subtyping subjects based on phenotypic information, comprising:
receiving a subject data unit for each of a plurality of subjects, each subject data unit representing a plurality of different phenotypic information items about the subject of the subject data unit;
using a deep learning algorithm to derive a lower dimensional representation of each subject data unit and a clustering algorithm to detect clusters of the resulting lower dimensional representations, each cluster representing a subtype of subjects that are phenotypically related to each other, wherein:
the deep learning algorithm and clustering algorithm are implemented by a single mathematical model in which the derivation of the lower dimensional representations and the detection of the clusters are performed jointly.

2. The method of claim 1, wherein the joint performance of the derivation of the lower dimensional representations and the detection of the clusters comprises optimizing a unified loss function having a term corresponding to the derivation of the lower dimensional representations and a term corresponding to the detection of the clusters.

3. The method of claim 2, wherein the unified loss function further comprises a regularization term.

4. The method of claim 1, wherein the single mathematical model is configured so that the clustering algorithm provides supervisory signals to the deep learning algorithm.

5. The method of claim 1, wherein the deep learning algorithm is an autoencoder based deep representation learning algorithm and the clustering algorithm is an unsupervised Gaussian Mixture Model clustering model.

6. The method of claim 1, wherein the subjects to be subtyped have Parkinson's disease and the detected clusters correspond to phenotypic subtypes of Parkinson's disease.

7. The method of claim 1, wherein the phenotypic information items comprise one or more of the following: blood markers, genetic data, clinical data, medical imaging data, demographic data.

8. The method of claim 1, wherein the phenotypic information items comprise blood test information with one or more of the following items: red blood cell count in blood, haematocrit level in blood, haemoglobin level in blood, mean cell volume in blood, platelet count in blood, white blood cell count in blood, Alk phos level in blood, urea level in plasma, estimated GFR in blood, sodium level in plasma, total bilirubin level in plasma, potassium level in plasma, alanine aminotransferase level in plasma, albumin level in plasma, mean cell haemoglobin level in blood, mean cell haemoglobin concentration in blood, basophil count in blood, creatinine level in plasma, lymphocyte count in blood, neutrophil count in blood, c-reactive protein level in plasma, monocyte count in blood, eosinophil count in blood.

9. The method of claim 1, comprising:
obtaining a further subject data unit comprising a plurality of different phenotypic information items about a subject to be assessed; and
using the single mathematical model to derive a lower dimensional representation of the subject data unit and assign the lower dimensional representation of the subject data unit to one of the detected clusters, thereby identifying to which of the clusters the subject to be assessed belongs.

10. The method of claim 1, further comprising:
performing one or more measurements to generate a respective one or more of the phenotypic information items represented by one or more of the subject data units.

11. A computer program comprising computer-readable instructions that cause a computer to perform the method of claim 1.

12. A computer program product storing the computer program of claim 11.

13. An apparatus for subtyping subjects based on phenotypic information, comprising:
a data receiving unit configured to receive a subject data unit for each of a plurality of subjects, each subject data unit representing a plurality of different phenotypic information items about the subject of the subject data unit; and
a data processing unit configured to:
use a deep learning algorithm to derive a lower dimensional representation of each subject data unit and a clustering algorithm to detect clusters of the resulting lower dimensional representations, each cluster representing a subtype of subjects that are phenotypically related to each other, wherein:
the deep learning algorithm and clustering algorithm are implemented by a single mathematical model in which the derivation of the lower dimensional representations and the detection of the clusters are performed jointly.

14. The device of claim 13, further comprising a sensor system configured to perform measurements on a subject or on a sample from a subject to provide one or more of the phenotypic information items about the subject.
